(19) United States
(12) Patent Application Publication
(10) Pub. No.: US 2024/0428919 A1
(43) Pub. Date: Dec. 26, 2024

(54) REHABILITATION SUPPORT SYSTEM, REHABILITATION SUPPORT METHOD, AND STORAGE MEDIUM

(71) Applicant: TOYOTA JIDOSHA KABUSHIKI KAISHA, Toyota-shi (JP)

(72) Inventors: Takuya IWATA, Toyota-shi (JP); Makoto KOBAYASHI, Nisshin-shi (JP)

(73) Assignee: TOYOTA JIDOSHA KABUSHIKI KAISHA, Toyota-shi (JP)

(21) Appl. No.: 18/667,001

(22) Filed: May 17, 2024

(30) Foreign Application Priority Data
Jun. 26, 2023 (JP) 2023-104037

(51) Int. Cl. G16H 20/30; G16H 10/60; G16H 50/70

(52) U.S. Cl. CPC G16H 20/30; G16H 10/60; G16H 50/70

(57) ABSTRACT

A rehabilitation support system includes: a patient information acquisition unit that acquires patient information relating to a target patient, the patient information including information of at least one reference time point; a related data acquisition unit that acquires related data of another patient similar to the patient information, the related data including training achievement data of rehabilitation, a transition of a recovery evaluation index, and information of reference time point corresponding to the reference time point of the target patient; a training plan creation unit that creates a training plan of the target patient based on the related data of the other patient by aligning the reference time point of the patient information and the reference time point of the other patient; and a presenting unit that presents the training plan.

### CROSS-REFERENCE TO RELATED APPLICATION

This application claims priority to Japanese Patent Application No. 2023-104037 filed on Jun. 26, 2023, incorporated herein by reference in its entirety.

### BACKGROUND

### 1. Technical Field

The present disclosure relates to a rehabilitation support system, a rehabilitation support method, and a storage medium.

### 2. Description of Related Art

In Japanese Unexamined Patent Application Publication No. 2015-47253, it is disclosed that part images to be displayed on an image display apparatus are added or changed in accordance with an achievement degree of rehabilitation exercises and a screen is updated as a result of changing aspects of the part images in accordance with the achievement degree of the rehabilitation exercises. Showing the screen to a person who is training can lead to maintenance of motivation.

### SUMMARY

A general patient develops symptoms (for example, develops cerebral infarction), undergoes surgery, is hospitalized in an acute period, and then starts rehabilitation in accordance with the recovery situation. Some patients also start rehabilitation after being transferred to another hospital in a recovery period. As above, the period of starting the rehabilitation differs depending on the medical condition, age, basic physical strength, and the like of the patient. There are also cases of a rehabilitation result in an acute period hospital and a rehabilitation result in a recovery period hospital. Thus, data of a hospitalized patient and data of a discharged patient of the past cannot be unconditionally compared with each other. Therefore, it is difficult to extract similar data by machine learning and the like and use the data of the past patient when a training plan of the hospitalized patient is created.

The present disclosure has been made in order to solve problems as above, and an object of the present disclosure is to provide rehabilitation support system, method, storage medium, and the like that support creation of a training plan of a patient with reference to training achievement data of another patient.

A rehabilitation support system according to one aspect of the present disclosure is a rehabilitation support system including: a patient information acquisition unit that acquires patient information relating to a target patient, the patient information including information of at least one reference time point; a related data acquisition unit that acquires related data of another patient similar to the patient information, the related data including training achievement data of rehabilitation, a transition of a recovery evaluation index, and information of reference time point corresponding to the reference time point of the target patient; a training plan creation unit that creates a training plan of the target patient based on the related data of the other patient by aligning the reference time point of the patient information and the reference time point of the other patient; and a presenting unit that presents the training plan.

A rehabilitation support method according to one aspect of the present disclosure is a rehabilitation support method including: acquiring patient information relating to a target patient, the patient information including information of at least one reference time point; acquiring related data of another patient similar to the patient information, the related data including training achievement data of rehabilitation, a transition of a recovery evaluation index, and information of reference time point corresponding to the reference time point of the target patient; creating a training plan of the target patient based on the related data of the other patient by aligning the reference time point of the patient information and the reference time point of the other patient; and presenting the training plan.

A storage medium storing a program according to one aspect of the present disclosure is a storage medium storing a program that causes a computer to execute: acquiring patient information relating to a target patient, the patient information including information of at least one reference time point; acquiring related data of another patient similar to the patient information, the related data including training achievement data of rehabilitation, a transition of a recovery evaluation index, and information of reference time point corresponding to the reference time point of the target patient; creating a training plan of the target patient based on the related data of the other patient by aligning the reference time point of the patient information and the reference time point of the other patient; and presenting the training plan.

According to the present disclosure, it becomes possible to create the training plan of the rehabilitation of the new patient with use of the achievement data of the other patient of the past.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a view showing an example of a user interface displayed by a rehabilitation support system;

FIG. 2 is a block diagram showing a configuration of the rehabilitation support system according to an embodiment;

FIG. 3 is a diagram showing a date of onset, an acute period, a recovery period, and a chronic period;

FIG. 4 is a flowchart showing an operation of the rehabilitation support system according to the embodiment;

FIG. 5 is a diagram showing an example of training achievement data of a discharged patient according to the embodiment;

FIG. 6 is a diagram showing an example of training achievement data of a hospitalized patient according to the embodiment;

FIG. 7 is a diagram showing an example that displays a training plan of the hospitalized patient according to the embodiment to be comparable with data of the discharged patient; and

FIG. 8 is a diagram showing a hardware configuration example of a training plan creation apparatus.

### DETAILED DESCRIPTION OF EMBODIMENTS

Embodiments of the present disclosure are described in detail below with reference to the drawings. In each drawing, the same or corresponding elements are denoted by the same reference characters, and overlapping descriptions are omitted as needed in order to clarify the description.

A rehabilitation support system of the present disclosure may be used to display various data of a patient that performs rehabilitation and to offer a more efficient rehabilitation support.

In a hospital, a therapist records rehabilitation training content and an amount of time required of the patient every predetermined period (for example, five minutes). The rehabilitation support system displays a rehabilitation achievement and a plan on a display and the like by utilizing record data accumulated for each patient.

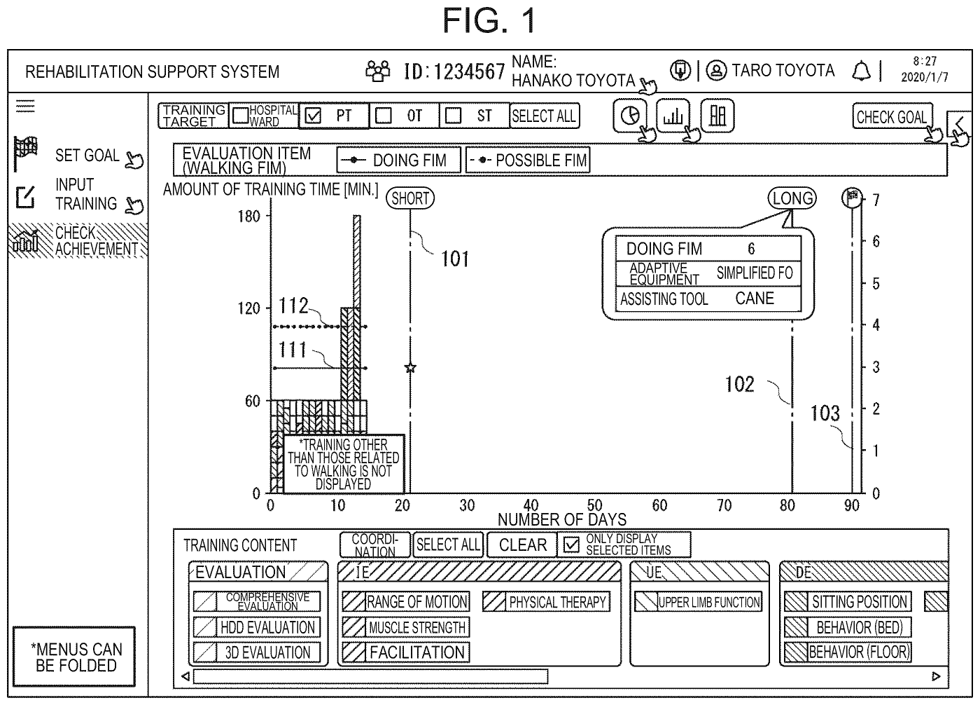

FIG. 1 is an example of a user interface displayed by the rehabilitation support system. In a central portion of a screen, a graph in which a horizontal axis is days and two vertical axes are an amount of training time and a recovery evaluation index is displayed. The right-side vertical axis in the graph expresses the amount of training time, and the left-side vertical axis in the graph expresses the recovery evaluation index. As a recovery evaluation index, a functional independence measure (FIM) is used, but it is not limited thereto, and appropriate indices such as a Barthel index (BI) and a stroke impairment assessment set (SIAS) that may be understood by a person skilled in the art may be used. As above, on the user interface, the user can check the training achievement and the recovery evaluation index side by side. The user interface may be displayed on a touch screen that can be operated by touching.

Evaluation data such as the training achievement and a walk FIM can be set by using day 0 in the horizontal axis as a date of onset. The date of onset is the day on which the symptom has started to appear. The user can also change day 0 in the horizontal axis to be an acute period hospitalization date, a recovery period hospitalization date, a rehabilitation start date, or the like instead of the date of onset by operating various buttons of the user interface.

The training achievement of the patient can be visualized on the user interface with use of a stacked bar graph or a line graph. The amount of training time for each training item of the patient recorded by the therapist who helps with the rehabilitation is displayed as the training achievement. In FIG. 1, a one-dot chain line 101 that is a short term goal is displayed where the number of training days in the horizontal axis is 20 days. A one-dot chain line 102 that is a long term goal is displayed where the number of training days in the horizontal axis is 80 days. A one-dot chain line 103 indicating an expected discharge date is also displayed where the number of training days in the horizontal axis is 90 days. Each of the lines of the short term goal, the long term goal, and the expected discharge date may be freely set by a doctor or the therapist or may be automatically set on the basis of the training achievement and the patient data of the past. Examples of the training items include 10 a walking training (for example, walking on level ground, stairs, and the like), an eating training, and a toilet training but are not limited thereto. The dates of the short term goal and the long term goal are not limited thereto and can be freely set.

The short term goal, the long term goal, or the expected discharge date can be set by the doctor or the therapist in consideration of the achievement, the motivation, the physical condition, and the like of the hospitalized patient with reference to data of other discharged patients similar to the hospitalized patient. The doctor or the therapist can appropriately communicate with the patient so as to raise the motivation for the rehabilitation by grasping the progress of achievement of the goal on the basis of the achievement data of the hospitalized patient with respect to the short term goal and the long term goal.

Each input data (for each training item) of the achievement data of the patient may be associated with a name or an ID of a therapist in charge. The user can selectively display the training achievement data associated with the therapist in charge by selecting the name of the therapist on the user interface. In some embodiments, the training achievement data associated with the therapist in charge may be displayed to be distinguishable by color coding and the like. The training achievement data can also be displayed to be distinguishable according to the type of the therapist. Examples of the types of therapists include a physical therapist (PT), an occupational therapist (OP), a speech therapist (ST), and the like but are not limited thereto. In some embodiments, an individual training in which the patient trains alone or a group training in which a plurality of patients gathers up and trains can also be distinguishably displayed. In the case of the individual training, the therapist asks the amount of time for each training item from the patient or a nurse and inputs the achievement data into the rehabilitation support system. In some embodiments, the achievement data for each training item may be automatically input to the rehabilitation support system from a sensor (for example, a wearable device) worn on the patient.

As shown in FIG. 1, a “doing FIM” 111 and a “possible FIM” 112 are displayed in association with the achievement data. The FIM expresses a recovery evaluation index. A maximum value (for example, 7) indicates that the patient can execute the training item as much as a healthy person, and the degree of necessity for nursing care is indicated to become gradually higher as the numerical value decreases.

The “doing FIM” evaluates what the patient is doing in life. For example, in the case of a walking training, when the patient can manage to walk without a cane but walks with a cane in everyday life, the “doing FIM” indicates that the patient can walk with a cane. In this case, the “possible FIM” indicates that it is possible to walk without a cane. The “doing FIM” can be acquired by regularly (for example, once a month) observing the daily life of the patient by the therapist. The “possible FIM” can be acquired by observing a daily rehabilitation state by the therapist. The “doing FIM” may be acquired for each of the time of hospitalization and the time of discharge, and a difference thereof may be used to evaluate the achievement of the rehabilitation.

The above is a general description relating to the user interface of the rehabilitation support system. As above, the data of various patients is accumulated and is recorded on a hospitalized patient and discharged patient database. However, the starting period of the rehabilitation differs depending on the medical condition, age, basic physical strength, and the like, and hence the data of the hospitalized patient and the data of the discharged patient of the past may not be able to be compared with each other. Therefore, it is difficult to use the data of the past when the training plan of the hospitalized patient is created. Thus, a reference time point of accumulated data of the past can be set in a selectable manner and can be used to create the training plan of the hospitalized patient by a method below.

### EXEMPLARY EMBODIMENTS

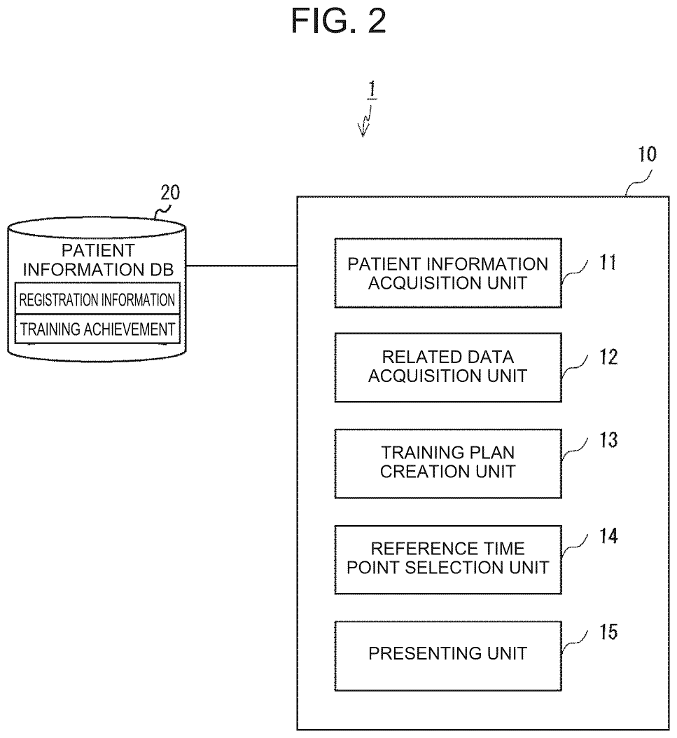

FIG. 2 is a block diagram showing a configuration of the rehabilitation support system according to an embodiment. A rehabilitation support system 1 includes a training plan presenting apparatus 10 and a discharged patient database 20. The training plan presenting apparatus 10 and the discharged patient database 20 are connected to each other via a network. In another embodiment, the presenting unit 15 may be provided separately from the training plan presenting apparatus 10. The training plan presenting apparatus 10 and the patient information database 20 may be integrally provided.

As described above, the discharged patient database 20 records training achievement data and recovery evaluation indices of the various patients from hospitalization to discharge and registration information (for example, name, sex, medical condition, and the like) of the patients.

The training plan presenting apparatus 10 is realized by a computer having a memory, a processor, a display, and the like. The training plan presenting apparatus 10 includes a patient information acquisition unit 11, a related data acquisition unit 12, a training plan creation unit 13, a reference time point selection unit 14, and a presenting unit 15.

The patient information acquisition unit 11 acquires patient information relating to a hospitalized patient. The patient information may include personal information of the patient (for example, name, age, sex, height, and weight) and information relating to a disease or a symptom of the patient (for example, cerebral infarction), for example. As described above, the patient information may include the training achievement of the rehabilitation, the recovery evaluation index (FIM), the date of onset, the hospitalization date, and the like input by a therapist via an operation unit (for example, a keyboard, a mouse, and the like).

The related data acquisition unit 12 acquires achievement data relating to another patient (for example, a discharged patient) similar to the hospitalized patient. The related data acquisition unit 12 may acquire achievement data relating to one or a plurality of discharged patients similar to the hospitalized patient. The other similar patient is a patient having a profile (registration information) such as age, sex, and medical condition that is similar to that of the hospitalized patient. For example, when the hospitalized patient is a 43 year old man having cerebral infarction, the related data acquisition unit 12 can acquire achievement data of other one or more patients that are men in their forties having cerebral infarction from the patient information database 20 as patients to which the hospitalized patient is similar. The achievement data of the other patients may also include information relating to the training achievement of the rehabilitation, the recovery evaluation index (FIM), the date of onset, hospitalization date, and the discharge date input by the therapist.

In some embodiments, the related data acquisition unit 12 acquires achievement data relating to discharged patients similar to a more specific training achievement or recovery evaluation index of the hospitalized patient. For example, when the hospitalized patient is a 43 year old man having cerebral infarction, achievement data relating to discharged patients similar to the training achievement or the recovery evaluation index of the hospitalized patient is acquired out of one or more achievement data of men in their forties having cerebral infarction. For example, by acquiring a recovery evaluation index of a discharged patient to which an inclination or a transition of the recovery evaluation index of the hospitalized patient is similar, a training plan described later can be made more accurately on the basis of the recovery evaluation index of the discharged patient. As similar data, the inclination or the transition of the recovery evaluation index of the hospitalized patient or the achievement data and the recovery evaluation index of the discharged patient within a threshold value can be acquired.

In some embodiments, data (for example, features of the FIM in the graph) of other one or more patients similar to the data of the hospitalized patient (for example, features of the FIM in the graph) may be extracted by a similarity identification technology using machine learning. In another embodiment, machine learning such as a support vector machine and a neural network may be used for inclination detection (image processing) of similar FIMs in both of the graphs.

As described above, the training plan creation unit 13 aligns a reference time point of achievement data of a discharged patient and a reference time point of data of a target hospitalized patient, and creates a training plan of the target hospitalized patient. In some embodiments, the reference time points of the discharged patient and the hospitalized patient may be the date of onset of each of the patients. By aligning the date of onset in the achievement data of the discharged patient and the date of onset of the hospitalized patient that is the same reference time point of the hospitalized patient, the training plan of the hospitalized patient can be created on the basis of the same reference time points.

In another embodiment, the reference time point of the discharged patient and the hospitalized patient may be a hospitalization date in a recovery period. In yet another embodiment, the reference time point of the discharged patient and the hospitalized patient may be a hospitalization date in an acute period. For example, in the case of a patient transferred from another hospital in a recovery period, a training plan of the hospitalized patient can be created by aligning a hospitalization date of the hospitalized patient and a hospitalization date of a patient in a recovery period of the past. In this case, the related data acquisition unit 12 may acquire data of other one or more patients transferred from another hospital in an acute period as the patient data of the past.

In the case of a patient transferred from another hospital in an acute period, a training plan of the hospitalized patient can be created by aligning a hospitalization date of the hospitalized patient and a hospitalization date of a patient in an acute period of the past. In this case, the related data acquisition unit 12 may acquire data of other one or more patients transferred from another hospital in an acute period as the patient data of the past.

As described above, the training plan creation unit 13 can automatically create an appropriate training plan by aligning the reference time point of the data of the discharged patient and the hospitalized patient.

In some embodiments, when training achievement data or recovery evaluation indices of a plurality of discharged patients are used, the training plan creation unit 13 may create a training plan of the hospitalized patient on the basis of an average value of the training achievement data or the recovery evaluation indices of the plurality of discharged patients. In the present specification, the training plan may include at least one or all of a scheduled end date or an expected discharge date, a short term goal date, a long term goal date, a goal amount of training time and items for a short term goal, a goal amount of training time and items for a long term goal, training items to be completed by the end of training, and a predicted amount of time required for each of the training items of the training plan.

In some embodiments, the training plan creation unit 13 can create a more accurate training plan with use of the training achievement and the recovery evaluation index of the hospitalized patient during several days after the hospitalization. For example, the training plan creation unit 13 can predict a date (short term goal date) by which a defined short term goal can be achieved on the basis of the achievement data of the discharged patient similar to the training achievement of the hospitalized patient up to the present. The training plan creation unit 13 can predict a date (long term goal date) by which a defined long term goal can be achieved or an expected discharge date on the basis of the achievement data of the discharged patient similar to the training achievement of the hospitalized patient up to the present. In another embodiment, when a training plan is created, the number of days off (for example, once a week or twice a week) desired by the hospitalized patient or the therapist can also be taken into consideration.

In another embodiment, the training plan creation unit 13 can specifically create a training plan including training items and an amount of training time that are necessary, for example, on the basis of a transition of a recovery evaluation index of a hospitalized patient similar to the transition of the recovery evaluation index of the hospitalized patient to date.

The reference time point selection unit 14 accepts an input from a therapist and the like via an input apparatus (for example, a keyboard, a touch screen, a mouse, and the like) regarding the achievement data of the discharged patient and the data of the hospitalized patient and selects one reference time point from a plurality of reference time points. The same reference time points are selected for the achievement data of the discharged patient and the data of the hospitalized patient in this case as well. For example, the reference time point may be selected from a group formed by the date of onset, the hospitalization date in the acute period, and the hospitalization date in the recovery period. This may be effective when the hospitalized patient is transferred to another hospital and a therapist of the new hospital cannot see the training achievement, the recovery evaluation index, and the like of the previous hospital. In some embodiments, the reference time point may include two reference time points, in other words, the date of onset and the hospitalization date in the acute period or the hospitalization date in the recovery period.

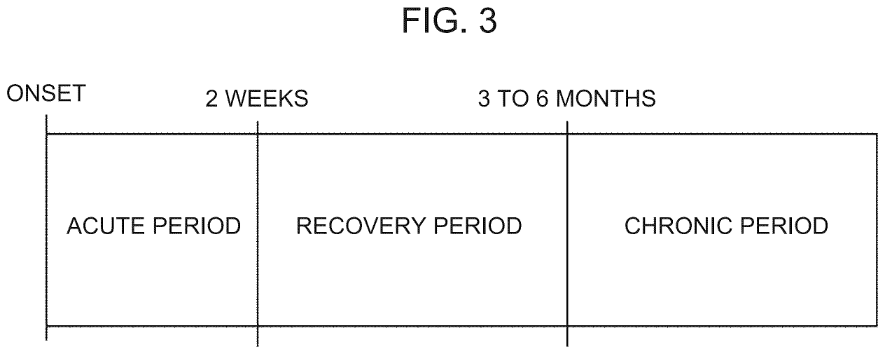

FIG. 3 is a diagram showing a date of onset, an acute period, a recovery period, and a chronic period. The training start date differs for each patient (for example, the training start date is in an acute period or a recovery period) and hence cannot be the reference time point. Therefore, in the present embodiment, the date of onset is the reference in principle when the patient data of the past and the data of the hospitalized patient are compared with each other. Two weeks starting from the date of onset is referred to as the acute period. Rehabilitation in the acute period is desired to start within basically 48 hours from the date of onset in order to prevent the decrease of physical functions. Therefore, many patients are hospitalized in the acute period and start rehabilitation. Some patients may exceptionally start rehabilitation after entering the recovery period. A period in which the patient lives at home after the state of the symptom and the disorder becomes stable is referred to as a chronic period. The number of days of the acute period or the recovery period in FIG. 3 is simply an exemplification and may differ for each medical condition.

The data of the hospitalized patient may be data that does not have any record of training achievement and indicates only the date of onset, for example. The data of the hospitalized patient may be data indicating the date of onset and the training achievement during several days or several tens of days from hospitalization. In some embodiments, the reference time point selection unit 14 sets the reference time point for comparing the achievement data of the discharged patient and the data of the hospitalized patient with each other to be the date of onset. In another embodiment, when the hospitalized patient starts the rehabilitation after entering a recovery period in which the condition becomes stable, the reference time point selection unit 14 may set the reference time point of the achievement data of the discharged patient to be the hospitalization date in the recovery period.

The presenting unit 15 presents the training plan created as described above on a display apparatus. The display apparatus may be a display of the training plan presenting apparatus 10 or may be a display outside of the training plan presenting apparatus 10. In some embodiments, the presenting unit 15 can present the training plan of the target patient to be comparable with the achievement data of the discharged patient.

Basically, the training plan of the hospitalized patient can be created by displaying two graphs in a comparing manner so as to cause the date of onset in the training achievement data of the discharged patient of the past and the date of onset in the training achievement data of the hospitalized patient to coincide with each other. In some embodiments, the two graphs of the discharged patient and the hospitalized patient may be displayed in an overlapping manner.

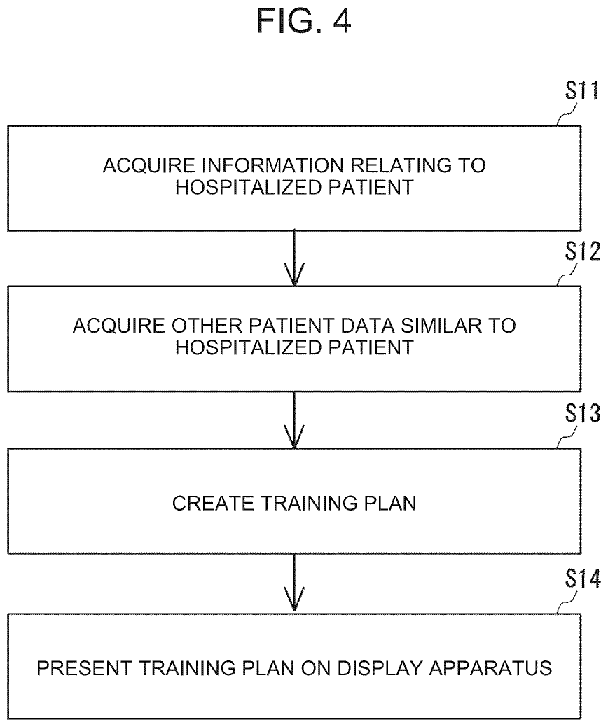

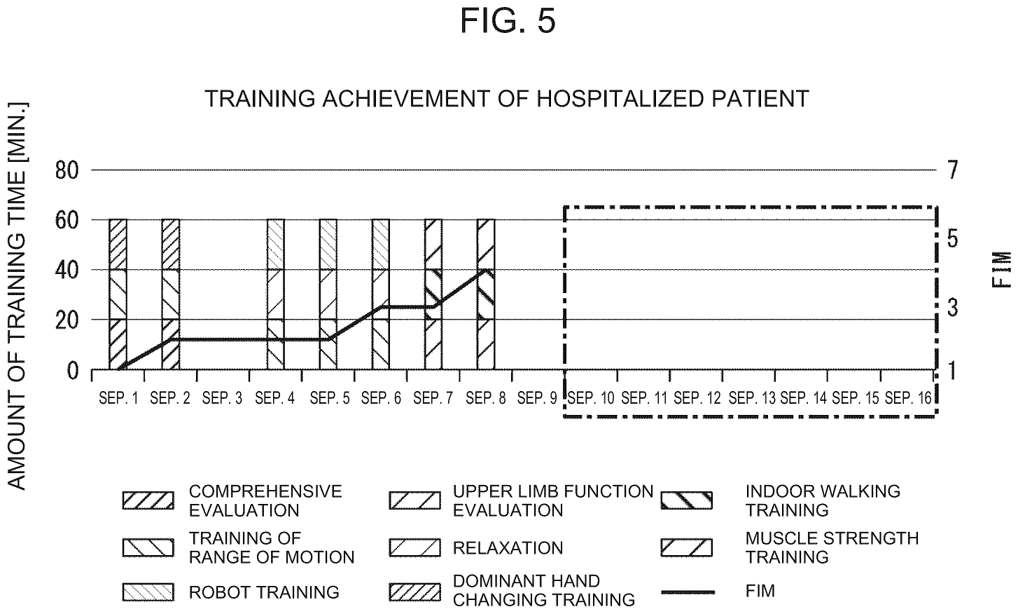

With reference to FIG. 4 to FIG. 7, a rehabilitation support method according to the embodiment is described. FIG. 4 is a flowchart showing an operation of the rehabilitation support system according to the embodiment. The patient information acquisition unit 11 acquires patient information relating to a hospitalized patient (Step S11). The patient information acquisition unit 11 accepts input operation from the therapist and acquires profile information of the patient such as the medical condition, the disease name (for example, cerebral infarction), the date of onset, and the hospitalization date of the patient. FIG. 5 shows a training achievement and a recovery evaluation index of a certain hospitalized patient. As shown in FIG. 5, the patient information acquisition unit 11 may acquire patient information including the training achievement of the rehabilitation and the recovery evaluation index of the patient. In the example of FIG. 5, the date of onset of the patient is August 31. The patient is hospitalized on September 1 and immediately starts the rehabilitation. In FIG. 5, training items and the amount of training time recorded from September 1 to September 8 are shown.

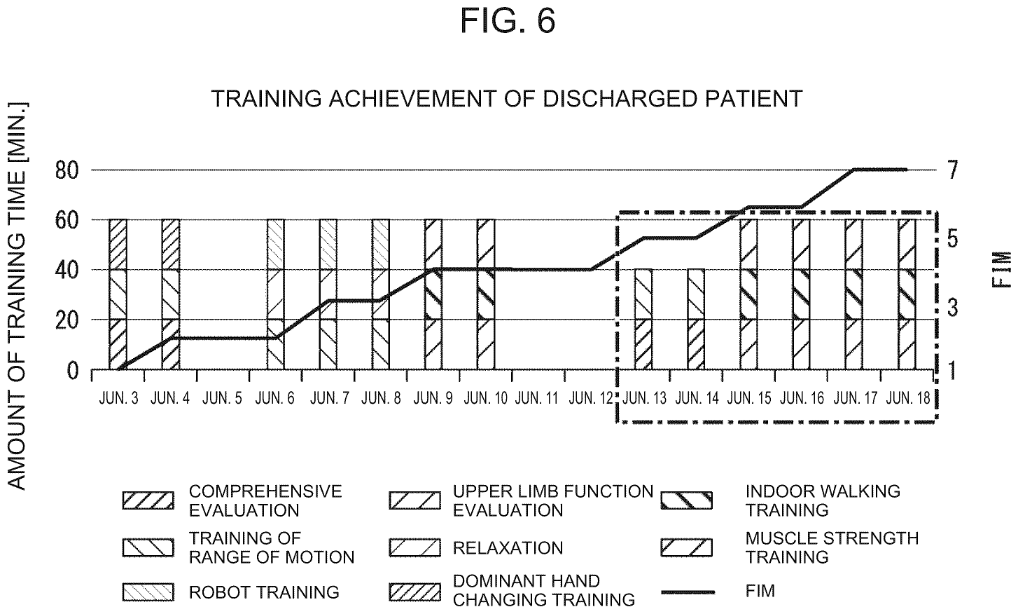

The related data acquisition unit 12 acquires achievement data relating to another patient (for example, a discharged patient) similar to the hospitalized patient (Step S12). For example, data of the past of other one or more discharged patients having the same medical condition (for example, cerebral infarction) as the hospitalized patient is acquired from the patient information database 20. In the present example, the related data acquisition unit 12 further acquires the training achievement and the recovery evaluation index of the hospitalized patient shown in FIG. 5 and data of another similar patient. Specifically, data in which a difference between an inclination of the recovery evaluation index of the hospitalized patient shown in FIG. 5 and an inclination of a recovery evaluation index of a discharged patient is smaller than a threshold value is extracted as similar data. FIG. 6 is an example showing the training achievement and the recovery evaluation index of the other patient that have been acquired. The other patient develops cerebral infarction on June 2, is hospitalized on June 3, and then starts rehabilitation as shown in FIG. 6. The other patient recovers to the same level (the FIM indicates 7) as a healthy person by June 17 and is discharged on June 18.

As described above, the training plan creation unit 13 aligns a reference time point of the achievement data of the other patient and a reference time point of the data of the hospitalized patient, and creates a training plan of the hospitalized patient (Step S13). In the present example, the training plan of the hospitalized patient is created by aligning the date of onset (June 2) in the achievement data of the other patient and the date of onset (August 31) in the data of the hospitalized patient. Specifically, the training plan of the hospitalized patient on September 9 and thereafter that does not have input record of achievement data is created. The training plan may be created with reference to the training achievement and the recovery evaluation index of the hospitalized patient to date, and the training achievement and the recovery evaluation index of the discharged patient of the past. The training plan may indicate that the expected discharge date is September 16.

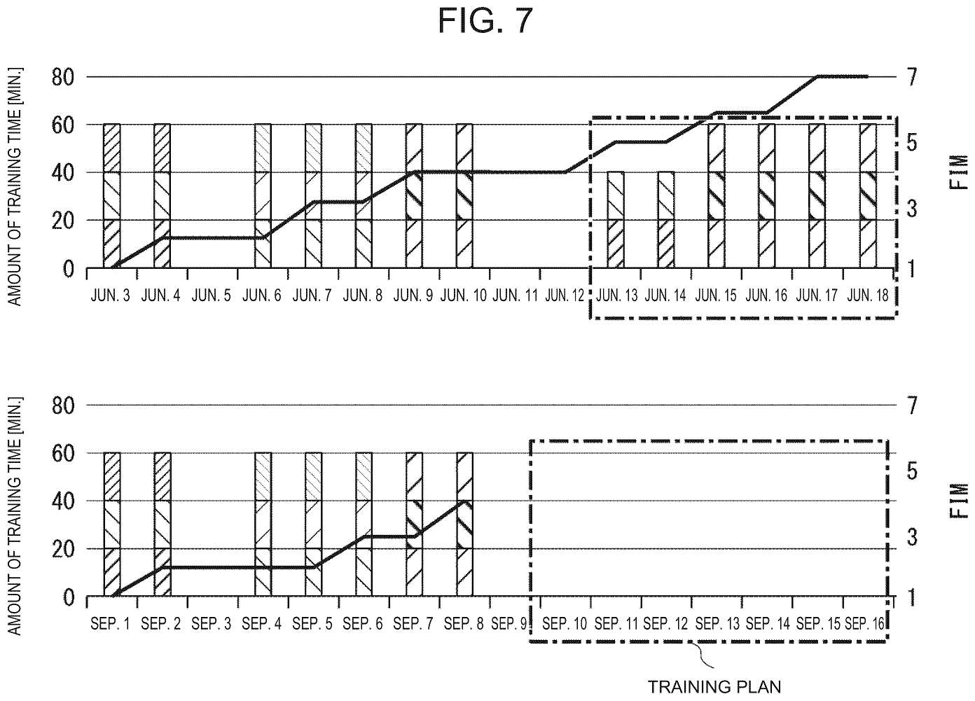

The presenting unit 15 presents the training plan created as described above (Step S14). FIG. 7 shows a display example of the training plan. The presenting unit 15 can present the training plan of the target patient (a lower diagram in FIG. 7) to be comparable with related data (an upper diagram in FIG. 7) of the discharged patient. The training plan indicates a scheduled end date or an expected discharge date (September 16 in the lower diagram in FIG. 7) of the training plan. In FIG. 7, the training plan is a blank space. However, in another embodiment, training items, the amount of training time, and the like necessary until the hospitalized patient is discharged may be specifically shown in the training plan. Alternatively, in another embodiment, a short term goal or a long term goal may be indicated in the training plan. The training plan does not necessarily need to be a comparable graph as above and may simply show the scheduled end date or the expected discharge date of the training plan, the training items and the amount of training time necessary until the hospitalized patient is discharged, and the like.

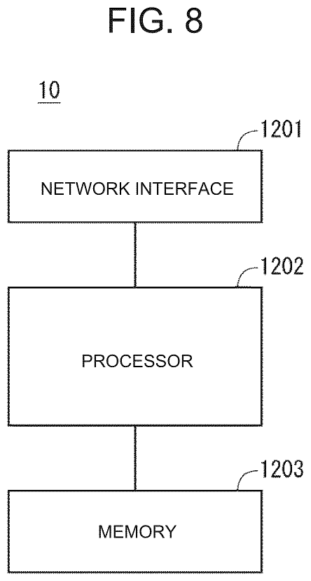

FIG. 8 is a block diagram showing a configuration example of the training plan creation apparatus 10. With reference to FIG. 8, the training plan creation apparatus 10 includes a network interface 1201, a processor 1202, and a memory 1203. The network interface 1201 is used to communicate with other network node apparatuses configuring a communication system. The network interface 1201 may be used to perform wireless communication. For example, the network interface 1201 may be used to perform wireless LAN communication defined in IEEE 802.11 series or mobile communication defined in 3rd Generation Partnership Project (3GPP (R)). Alternatively, the network interface 1201 may include a network interface card (NIC) that complies with IEEE 802.3 series, for example.

The processor 1202 performs processing of the training plan creation apparatus 10 described with use of a flowchart or a sequence in the embodiment described above by reading out and executing software (computer program) from the memory 1203.

The processor 1202 may be a microprocessor, a micro processing unit (MPU), a central processing unit (CPU), or a graphics processing unit (GPU), for example. The processor 1202 may include a plurality of processors.

The memory 1203 is configured by a combination of a volatile memory and a non-volatile memory. The memory 1203 may include a storage disposed to be spaced apart from the processor 1202. In this case, the processor 1202 may access the memory 1203 via an I/O interface (not shown).

In the example in FIG. 8, the memory 1203 is used to store software module groups therein. The processor 1202 can perform the processing of the training plan creation apparatus 10 described in the embodiment described above by reading out and executing those software module groups from the memory 1203.

As described with reference to FIG. 4, each of the processors included in the training plan creation apparatus 10 and the like executes one or a plurality of programs including an instruction group for causing a computer to execute algorithms described with reference to the drawings.

In the example described above, the program can be stored with use of various types of non-transitory computer readable media and can be supplied to a computer. The non-transitory computer readable media include various types of tangible recording media (storage media). Examples of the non-transitory computer readable media include a magnetic recording medium (for example, a flexible disk, a magnetic tape, and a hard disk drive), a magneto-optical recording medium (for example, a magneto-optical disk), a CD-ROM (read only memory), a CD-R, a CD-R/W, a digital versatile disc (DVD), a Blu-ray (R) disc (BD), and a semiconductor memory (for example, a mask ROM, a programmable ROM (PROM), an erasable PROM (EPROM), a flash ROM, and a random access memory (RAM)). The program may be supplied to the computer by various types of transitory computer readable media. Examples of the transitory computer readable media include electric signals, optical signals, and electromagnetic waves. The transitory computer readable medium can supply the program to the computer via wired communication paths such as an electric wire and an optical fiber or wireless communication paths.

The present disclosure is not limited to the embodiments described above and can be changed, as appropriate, without departing from the gist.

What is claimed is:

1. A rehabilitation support system, comprising:
a patient information acquisition unit that acquires patient information relating to a target patient, the patient information including information of at least one reference time point;
a related data acquisition unit that acquires related data of another patient similar to the patient information, the related data including training achievement data of rehabilitation, a transition of a recovery evaluation index, and information of reference time point corresponding to the reference time point of the target patient;
a training plan creation unit that creates a training plan of the target patient based on the related data of the other patient by aligning the reference time point of the patient information and the reference time point of the other patient; and
a presenting unit that presents the training plan.

2. The rehabilitation support system according to claim 1, wherein a plurality of the reference time points includes a date of onset, and a hospitalization date in an acute period or a hospitalization date in a recovery period.

3. The rehabilitation support system according to claim 1, wherein the presenting unit presents the training plan of the target patient to be comparable with the related data of the other patient.

4. The rehabilitation support system according to claim 1, further comprising a reference time point selection unit that selects a reference time point of data from a group formed by a date of onset, a hospitalization date in an acute period, and a hospitalization date in a recovery period.

5. The rehabilitation support system according to claim 1, wherein the patient information acquisition unit acquires a disease name, a training achievement, a recovery evaluation index, a date of onset, and a hospitalization date relating to the target patient.

6. The rehabilitation support system according to claim 1, wherein the related data acquisition unit acquires the related data of the other patient to which the transition of the recovery evaluation index of the target patient is similar by machine learning.

7. The rehabilitation support system according to claim 1, wherein the training plan creation unit creates the training plan of the target patient based on the related data of the other patient to which the transition of the recovery evaluation index of the target patient is similar.

8. The rehabilitation support system according to claim 1, wherein the training plan includes at least one of a scheduled end date, a short term goal date, a long term goal date, training items to be completed by an end of training, and a predicted amount of time required for each of the training items of the training plan.

9. A rehabilitation support method, comprising:
acquiring patient information relating to a target patient, the patient information including information of at least one reference time point;
acquiring related data of another patient similar to the patient information, the related data including training achievement data of rehabilitation, a transition of a recovery evaluation index, and information of reference time point corresponding to the reference time point of the target patient;
creating a training plan of the target patient based on the related data of the other patient by aligning the reference time point of the patient information and the reference time point of the other patient; and
presenting the training plan.

10. A non-transitory storage medium storing a program that causes a computer to execute:
acquiring patient information relating to a target patient, the patient information including information of at least one reference time point;
acquiring related data of another patient similar to the patient information, the related data including training achievement data of rehabilitation, a transition of a recovery evaluation index, and information of reference time point corresponding to the reference time point of the target patient;
creating a training plan of the target patient based on the related data of the other patient by aligning the reference time point of the patient information and the reference time point of the other patient; and
presenting the training plan.
